(19) United States
(12) Patent Application Publication
(10) Pub. No.: US 2021/0140926 A1
(43) Pub. Date: May 13, 2021

(54) RESTITUTION COEFFICIENT MEASURING DEVICE AND HARDNESS MEASURING DEVICE

(71) Applicant: Mitutoyo Corporation, Kawasaki (JP)

(72) Inventors: Ryu NUMASATO, Kure (JP); Masaru KAWAZOE, Kure (JP)

(73) Assignee: Mitutoyo Corporation, Kawasaki (JP)

(21) Appl. No.: 17/093,846

(22) Filed: Nov. 10, 2020

(30) Foreign Application Priority Data
Nov. 12, 2019 (JP) 2019-205094

(51) Int. Cl. G01N 29/04; G01N 3/52

(52) U.S. Cl. CPC G01N 29/045; G01N 2033/008; G01N 3/52

(57) ABSTRACT

A restitution coefficient measuring device measuring a restitution coefficient of a measuring object includes a holder that holds a spherical impact ball colliding with the measuring object with an elastic member, a shooting mechanism for shooting the impact ball held by the holder from the holder toward the measuring object, a speed measuring unit that measures both a collision speed at which the impact ball collides with the measuring object and a restitution speed at which the impact ball bounces from the measuring object, and a computing unit that calculates the restitution coefficient on the basis of the restitution speed with respect to the collision speed. A hole for bleeding air is bored on any side surface of the holder. The elastic member is an independent member replaceable with respect to the holder and is disposed at an end portion of the holder in an axial direction.

### BACKGROUND OF THE INVENTION

### Field of the Invention

The present invention relates to a restitution coefficient measuring device measuring a restitution coefficient of a specimen by causing an impact ball to collide with the specimen, and a hardness measuring device measuring a hardness of the specimen.

### Description of the Related Art

In the related art, a measuring device in which an impact ball is shot toward a specimen and a restitution coefficient or a hardness of the specimen is measured from a ratio of a speed immediately before a collision of this impact ball to a speed immediately after the collision and a restitution is known (for example, refer to Japanese Patent Laid-open No. H10-239230 and Japanese Patent Laid-open No. 2017-90432).

Japanese Patent Laid-open No. H10-239230 discloses improvement in a method in which a speed of an indenter hammer serving as an impact ball is detected using a hardness meter.

In addition, Japanese Patent Laid-open No. 2017-90432 discloses that when an indenter serving as an impact ball is shot toward a specimen, the indenter is held at a front end of a holder. Slits extending in a manner of being parallel to an axis are formed at the front end of the holder, which is constituted of a plurality of split portions. Therefore, this holder can hold an outer circumferential surface of the indenter with the plurality of split portions.

In a measuring device measuring a restitution coefficient or a hardness of a specimen, an impact ball is stably shot at a predetermined speed, and thus the accuracy of a measurement value obtained by measuring the restitution coefficient or the hardness of the specimen can be enhanced.

In contrast, in the invention described in Japanese Patent Laid-open No. H10-239230, there is no disclosure regarding details of a mechanism for shooting an indenter hammer, and there is room for improvement in stably shooting the impact ball.

In addition, the invention described in Japanese Patent Laid-open No. 2017-90432 discloses that a spherical indenter is held by a plurality of split portions at a front end of a holder. However, for example, it is conceivable that a holding force for holding the indenter may vary due to a slight difference between the dimensions of inner diameters of the plurality of split portions. Accordingly, the dimensions and the opening degrees of the plurality of split portions need to be delicately adjusted at the time of assembly, and there is room for improvement therein.

### SUMMARY OF THE INVENTION

Embodiments of the present invention provide a restitution coefficient measuring device in which stable shooting of an impact ball is improved, and a hardness measuring device.

According to an embodiment of the present invention, there is provided a restitution coefficient measuring device measuring a restitution coefficient of a measuring object. The restitution coefficient measuring device includes a holder that holds a spherical impact ball colliding with the measuring object with an elastic member, a shooting mechanism for shooting the impact ball held by the holder from the holder toward the measuring object, a speed measuring unit that measures both a collision speed at which the impact ball collides with the measuring object and a restitution speed at which the impact ball bounces from the measuring object, and a computing unit that calculates the restitution coefficient on the basis of the restitution speed with respect to the collision speed. A hole for bleeding air is bored on any side surface of the holder. The elastic member is an independent member replaceable with respect to the holder and is disposed at an end portion of the holder in an axial direction.

Further features of the present invention will become apparent from the following description of exemplary embodiments with reference to the attached drawings.

### BRIEF DESCRIPTION OF THE DRAWINGS

FIG. 1 is a side cross-sectional view showing a structure of a restitution coefficient measuring device according to Example 1 of the present invention.

FIG. 2 is an enlarged side cross-sectional view showing a part in the vicinity of an end portion of a holder 5 on one side in the axial direction in FIG. 1.

FIG. 3 is a cross-sectional view of the holder 5 along III-III in FIG. 2.

FIG. 4 is an enlarged side cross-sectional view showing a part in the vicinity of an engagement member 23 of a shooting mechanism 3 shown in FIG. 1.

FIG. 5 is a cross-sectional view along V-V in FIG. 4.

FIG. 6 is an enlarged side cross-sectional view showing a part in the vicinity of the engagement member 23 of the shooting mechanism 3 shown in FIG. 1.

FIG. 7 is a cross-sectional view along VII-VII in FIG. 6.

FIG. 8 is an enlarged side cross sectional view showing a part in the vicinity of an end portion of a holder 105 corresponding to the holder 5 in FIG. 1 on one side in the axial direction in Example 2.

FIG. 9 is a cross-sectional view of the holder 105 along IX-IX in FIG. 8.

FIG. 10 is an enlarged side cross-sectional view showing a pad in the vicinity of an end portion of a holder 205 corresponding, to the holder 5 in FIG. 1 on one side in the axial direction in Example 3.

FIG. 11 is a cross-sectional view of the holder 205 along XI-XI in FIG. 10.

FIG. 12 is an enlarged side cross-sectional view showing a part in the vicinity of an end portion of a holder 305 corresponding to the holder 5 in FIG. 1 on one side in the axial direction in Example 4.

FIG. 13 is a cross-sectional view of the holder 305 along XIII-XIII in FIG. 12.

FIG. 14 is an enlarged side cross-sectional view showing a part in the vicinity of an end portion of a holder 405 corresponding to the holder 5 in FIG. 1 on one side in the axial direction in Example 5.

FIG. 15 is a cross-sectional view of the bolder 405 along, XV-XV in FIG. 14.

FIG. 16 is a cross-sectional view showing the holder 405 along XV-XV in which an impact ball 6 and an, elastic member 420 are deleted from FIG. 15.

FIG. 17 is an enlarged side cross-sectional view showing a part in the vicinity of an end portion of a holder 505 corresponding to the holder 5 in FIG. 1 on one side in the axial direction in Example 6.

FIG. 18 is a cross-sectional view of the holder 505 along XVIII-XVIII in FIG. 17.

FIG. 19 is a plan view of the holder 505 in FIG. 17 viewed from above.

FIG. 20 is an enlarged side cross-sectional view showing a part in the vicinity of an end portion of a holder 605 corresponding to the holder 5 in FIG. 1 on one side in the axial direction in Example 7.

FIG. 21 is a cross-sectional view of the bolder 605 along XXI-XXI in FIG. 20.

FIG. 22 is an enlarged side cross-sectional view showing a part in the vicinity of an end portion of a holder 705 corresponding to the holder 5 in FIG. 1 on one side in the axial direction in Example 8.

FIG. 23 is a cross-sectional view of the holder 705 along XXIII-XXIII in FIG. 22.

FIG. 24 is an enlarged side cross-sectional view showing a part in the vicinity of an end portion of a holder 805 corresponding to the holder 5 in FIG. 1 on one side in the axial direction in Example 9.

FIG. 25 is a cross-sectional view of the holder 805 along XXV-XXV in FIG. 24.

### DESCRIPTION OF THE EMBODIMENTS

Hereinafter, apparatuses, that is, a restitution coefficient measuring device and a hardness measuring device according to the present invention will be described in detail with reference to the drawings. In the following, drawings, in order to facilitate the understanding of each configuration, the scales, the numbers, and the like of an actual structure and each structure may vary.

In addition, a direction parallel to a central axis J of a tube hole 5b (refer to FIG. 2) of a tubular holder 5 shown in FIG. 1 will be simply referred to as “an axial direction”. The lower side of FIG. 1 in the axial direction, that is, a side close to a specimen 8 will be referred to as one side in the axial direction. The upper side of FIG. 1 in the axial direction, that is, a side away from the specimen 8 will be referred to as the other side in the axial direction. In addition, a radial direction centering on the central axis will be simply referred to as “a radial direct on”. A direction around an axis centering on the central axis J will be simply referred to as “a circumferential direction”.

The axial direction, the radial direction, the circumferential direction, the upper side, the lower side, the right side, and the let side are names for simply describing the relative positional relationship of each past, and the actual disposition relationship or the like may be a disposition relationship or the like other than the disposition relationships or the like indicated by these names. In addition, in this specification, forward, rearward, left, right, upward, and downward directions and the like indicate directions viewed in the drawings and they do not limit the directions at the time of using the apparatuses according to the present invention.

In this specification, the expression “extending, in the axial direction or the radial direction” also includes a case of extending in a direction tilted within a range less than 45° with respect to the axial direction or the radial direction, in addition to a case of strictly extending in the axial direction or the radial direction.

### Example 1

(Structure of Restitution Coefficient Measuring Device 1)

FIG. 1 is a side cross-sectional view showing a structure of a restitution coefficient measuring device according to Example 1 of the present invention.

A restitution coefficient measuring device 1 includes a holder 5 that holds an impact ball 6 serving as a spherical indenter, a shooting mechanism 3 for shooting the impact ball 6 held by the holder 5 from the holder 5 toward the specimen 8, a speed measuring unit 12 that measures a collision speed which is a speed of the impact ball 6 immediately before the impact ball 6 collides with the specimen 8 and a restitution speed which is a speed of the impact ball 6 after the impact ball 6 has collided with the specimen 8 and bounced (restituted), and a computing unit 10 that calculates a restitution coefficient which is a ratio of the restitution speed to the collision speed. The computing unit 10 is included inside a display 9 having a display unit 11 displaying the restitution coefficient calculated by the computing unit 10. The impact ball 6 is made of a ceramic, for example. The impact ball 6 is made of a cemented carbide, for example.

The shooting mechanism 3 has a shooting member 4. The shooting member 4 is disposed inside the tube hole 5b of the holder 5. The shooting mechanism 3 shoots the impact ball 6 to one side in the axial direction by moving the shooting member 4 to one side in the axial direction and causing the shooting r member 4 to collide with the impact ball 6. The shooting mechanism 3 has a energizing portion 2 energizing the shooting member 4 to one side in the axial direction. The energizing portion 2 has an elastic member energizing the shooting member 4 to one side in the axial direction. This elastic member is a coil spring, for example. The energizing portion 2 is not limited to a portion energizing the shooting member 4 with an elastic member and may be a portion energizing an elastic member with air, for example. The collision speed which is a speed of the impact ball 6 immediately before the impact ball 6 collides with the specimen 8 can be adjusted by adjusting a energizing force of the energizing portion 2. The shooting mechanism 3 has an engagement member 23 (refer to FIG. 4) detaining the shooting member 4 at an initial position. FIG. 1 shows a state in which the shooting member 4 is detained at the initial position. The shooting mechanism 3 moves the shooting member 4 to one side in the axial direction by disengaging the engagement member 23 from the shooting member 4. A mechanism in which the shooting mechanism 3 moves the shooting member 4 to one side in the axial direction will be described below in detail with reference to FIGS. 4 to 7.

The holder 5 has a tubular shape. The holder 5 has a cylindrical shape, for example. The impact ball 6 is shot toward one side in the axial direction along the central axis J of a tube hole of the holder 5. The holder 5 is disposed inside a tube hole of a tube member 16 having a tubular shape. The tube member 16 has a cylindrical shape, for example. The speed measuring unit 12 has a penetration hole which is disposed on one side of the tube member 16 in the axial direction and penetrates the speed measuring unit 12 from the tube member 16 side to the specimen 8 side. A ball path 13 through which the impact ball 6 shot by the shooting mechanism 3 passes is formed by the tube hole of the tube member 16 and the penetration hole of the speed measuring unit 12.

The speed measuring unit 12 includes a speed measuring main body 7 in which the penetration hole forming the ball path 13 is formed, and a first passage sensor 15 and a second passage sensor 14 which are arranged along the penetration hole. In the first passage sensor 15 and the second passage sensor 14, the first passage sensor 15 is disposed on one side in the axial direction from the second passage sensor 14. The passage sensor is a generic name of a sensor capable of detecting passage of an object. This passage sensor includes an optical sensor and a magnetic sensor, for example.

The first passage sensor 15 of the present example is an optical sensor having a first irradiation unit 15a irradiating the inside of the penetration hole forming the ball path 13 with light, and a first light receiving unit 15b receiving irradiation light from the first irradiation unit 15a. The second passage sensor 14 of the present example is an optical sensor having a second irradiation unit 14a irradiating the inside of the penetration hole forming the ball path 13 with light, and a second light receiving unit 14b receiving irradiation light from the second irradiation unit 14a. The speed measuring unit 12 detects that the impact ball 6 has passed through the position of the first passage sensor 15 by detecting interruption of light received by the first light receiving unit 15b and detects that the impact ball 6 has passed through the position of the second passage sensor 14 by detecting interruption of light received by the second light receiving unit 14b.

The computing unit 10 can obtain the collision speed from a difference between a time when the impact ball 6 passes through the position of the second passage sensor 14 and a time when the impact ball 6 passes through the position of the first passage sensor 15 thereafter and a difference between the position of the second passage sensor 14 and the position of the first passage sensor 15. The computing unit 10 can obtain the restitution speed from a difference between a time when the impact ball 6 passes through the position of the first passage sensor 15 again after the impact ball 6 has passed through the position of the first passage sensor 15 and a time when the impact ball 6 passes through the position of the second passage sensor 14 thereafter, and a difference between the position of the first passage sensor 15 and the position of the second passage sensor 14.

(Structure of Holder 5)

FIG. 2 is an enlarged side cross-sectional view showing a part in the vicinity of an end portion of the holder 5 on one side in the axial direction in FIG. 1. In the illustration of FIG. 1, the other side in the axial direction is disposed on the upper side and one side in the axial direction is disposed on the lower side. However, in the illustration of FIG. 2, the other side in the axial direction is disposed on the right side and one side in the axial direction is disposed on the left side.

FIG. 3 is a cross-sectional view of the holder 5 along III-III in FIG. 2.

The restitution coefficient measuring device 1 of the present example has an elastic member 20 which is a member independent from the holder 5. The elastic member 20 is made of an elastic material such as a rubber or a metal, for example, and the material thereof does not natter particularly. The elastic member 20 is an annular member. In the present example, the elastic member 20 is a cylindrical member. In the present example, the elastic member 20 has a seamlessly annular O-shape, but the elastic member 20 may have a disconnected annular C-shape. The diameter of the tube hole of the elastic member 20 is smaller than the diameter of the impact ball 6, and the impact ball 6 is held in the tube hole of the elastic member 20. The elastic member 20 has a thin wall portion 20a having a larger inner diameter than that on one side in the axial direction at an end portion on the other side in the axial direction. The holder has a thin wall portion 5a having a smaller outer diameter than that on the other side in the axial direction at an end portion on one side in the axial direction. The thin wall portion 20a of the elastic member 20 is fitted to the thin wall portion 5a of the holder 5 on a side outward in the radial direction. According to the present example, the impact ball 6 can be held by means of only the elasticity of the material of the elastic member 20. When a holding force for holding the impact ball 6 becomes weak, the holding force for holding the impact ball 6 can be recovered simply by replacing the elastic member 20.

The holder 5 has a penetration hole 5c penetrating the holder 5 from a side outward in the radial direction to a side inward in the radial direction on the other side in the axial direction from the impact ball 6 and one side in the axial direction from the shooting member 4. When the shooting member 4 moves to one side in the axial direction, air between the shooting member 4 and the impact ball 6 is compressed. For this reason, when the penetration hole 5c is not provided, there is a possibility that the impact ball 6 may be pressed by compressed air and come off from the bolder 5. According to the present example, air between the shooting member 4 and the impact ball 6 can escape to the outside through the penetration hole 5c. Therefore, the impact ball 6 is prevented from being pressed by compressed air and coming off from the bolder 5, and thus shooting of the impact ball 6 can be limited to shooting by means of a collision of the shooting member 4. From this, according to the present example, the impact ball 6 can be shot at a predetermined stable shooting speed.

(Structure of Shooting Mechanism 3)

FIG. 4 is an enlarged side cross-sectional view showing a part in the vicinity of the engagement member 23 of the Shooting mechanism 3 shown in FIG. 1. FIG. 5 is a cross-sectional view along V-V in FIG. 4. FIGS. 4 and 5 are views showing a state in which the engagement member 23 engages with the shooting member 4 and the shooting member 4 is detained. FIG. 6 is an enlarged side cross-sectional view showing a part in the vicinity of the engagement member 23 of the shooting mechanism 3 shown in FIG. 1. FIG. 7 is a cross-sectional view along VII-VII in FIG. 6. FIGS. 6 and 7 are views showing a state in which the shooting member 4 is disengaged from the engagement member 23. The engagement member 23 is an example of a restriction portion restricting movement of the shooting member 4 to one side in the axial direction.

The shooting member 4 has a small diameter portion 4b on one side in the axial direction, a large diameter portion 4a having a larger diameter than the small diameter portion 4b on the other side in the axial direction from the small diameter portion 4b, and a stepped portion 4c which is a step generated due to a difference between the diameters of the small diameter portion 4b and the large diameter portion 4a.

In the present example, the tube member 16 has a hole 16b penetrating the tube member 16 from a side outward in the radial direction (right side in FIG. 4) to a tube hole 16a and is recessed from an inner wall of the facing tube hole 16a toward a side outward in the radial direction (left side in FIG. 4). The engagement member 23 is inserted into the hole 16b through an opening of the hole 16b on a side outward in the radial direction (right side in FIG. 4). Here, the right side in FIG. 4 will be referred to as the other side in the radial direction, and the left side in FIG. 4 will be referred to as one side in the radial direction. After the engagement member 23 is inserted into the hole 16b, a frame member 24 is fitted into the opening of the hole 16b on the other side in the radial direction. The engagement member 23 can move inside the hole 16b in the radial direction. After the frame member 24 is fitted into the opening of the hole 16b on the other side in the radial direction, a button 21 is fixed to an end portion of the engagement member 23 on the other side in the radial direction. An elastic member 22 is provided between the frame member 24 and the button 21, and the elastic member 22 biases the button 21 to the other side in the radial direction. The elastic member 22 is a coil spring, for example. An operator can press down the button 21 to one side in the radial direction, for example. When no operation is applied from the outside, the button 21 moves to the other side in the radial direction due to a energizing force of the elastic member 22.

The engagement member 23 has an engagement portion 23a on one side in the radial direction. The engagement portion 23a has a penetration hole 23b penetrating the engagement portion 23a in the axial direction. In the present example, as shown in FIG. 5, the shape of the penetration hole 23b viewed in the axial direction is an elliptical shape. FIGS. 4 and 5 are views showing a state when no operation is applied to the button 21 from the outside. When the button 21 moves to the other side in the radial direction, the engagement member 23 fixed to the button 21 also moves to the other side in the radial direction due to a energizing force of the elastic member 22. At this time, an end portion of the penetration hole 23b an one side in the radial direction comes into contact with an outer circumference of the small diameter portion 4b of the shooting member 4, and the stepped portion 4c engages with an edge of the penetration 23b, thereby locking the shooting member 4 at the initial position.

FIGS. 6 and 7 are views showing a state when the button 21 is pressed down to one side in the radial direction. The penetration hole 23b is larger than the large diameter portion 4a of the shooting member 4. In the state shown in FIGS. 6 and 7, the edge of the penetration hole 23b does not overlap the large diameter portion 4a in the axial direction. For this reason, the shooting member 4 moves to one side in the axial direction due to a energizing force of the energizing portion 2 and collides with the impact ball 6, thereby shooting the impact ball 6.

### Example 2

(Structure of Holder 105)

Example 2 shows another example of the structure of the end portion of the holder 5 on one side in the axial direction in FIG. 1. The present example is the same as Example 1 except for the structure of an end portion of a holder 105 on one side in the axial direction, and thus detailed description will be on omitted.

FIG. 8 is an enlarged side cross-sectional view showing a part in the vicinity of the end portion of the holder 105 corresponding to the holder 5 in FIG. 1 on one side in the axial direction in Example 2.

FIG. 9 is a cross-sectional view of the holder 105 along IX-IX in FIG. 8.

The restitution coefficient measuring device 1 of the present example has an elastic member 120 which is a member independent from the bolder 105. The elastic member 120 is made of an elastic material such as a rubber or a metal, for example, and the material thereof does not matter particularly. The elastic member 120 is an annular member. In the present example, the elastic member 120 is a tonic member. In the present example, the elastic member 120 has a seamlessly annular O-shape, but the elastic member 120 may have a disconnected annular C-shape. The bolder 105 is a tubular member and has a tube hole 105b. The holder 105 has a hole 105e extending in the axial direction at an end portion on one side in the axial direction. An inner diameter of the hole 105e is larger than an inner diameter of the tube hole 105b. The inner diameter of the hole 105e is larger than the diameter of the impact ball 6. The inner diameter of the tube hole 105b is smaller than the diameter of the impact ball 6. The holder 105 has a tapered portion 105d gradually decreasing in size from the inner diameter of the hole 105e larger than the diameter of the impact ball 6 to the inner diameter of the tube hole 105b smaller than the diameter of the impact ball 6 from one side in the axial direction toward the other side in the axial direction. The impact ball 6 inserted from one side of the holder 105 in the axial direction comes into contact with the tapered portion 105d, thereby being subjected to positioning. The tapered portion 105d is an example of a positioning portion performing positioning of the impact ball 6. A positioning portion other than the tapered portion 105d may be configured to protrude in the bole 105e to a side inward in the radial direction.

The bolder 105 has a groove portion 105f recessed to a side outward in the radial direction in the hole 105e. An outer circumference of the elastic member 120 is fitted into the groove portion 105f. An inner diameter of the elastic member 120 fitted into the groove portion 105f is smaller than the diameter of the impact ball 6, and the impact ball 6 is held by the elastic member 120. The impact ball 6 comes into contact with the elastic member 120 throughout the whole circumference of an inner circumference of the elastic member 120. According to the present example, a holding position and a holding force of the impact ball 6 can be managed by the dimensions of the elastic member 120. In addition, the tapered portion 105d is used as a stopper of the impact ball 6 so that the holding position of the impact ball 6 and a shooting distance of the impact ball 6 can be made uniform every time a shooting operation is performed. In addition, when a holding force for holding the impact ball 6 becomes weak, the holding force for holding the impact ball 6 can be recovered simply by replacing the elastic member 120.

The holder 105 has a penetration hole 105c penetrating the holder 105 from a side outward in the radial direction to a side inward in the radial direction on the other side in the axial direction from the impact ball 6 and one side in the axial direction from the shooting member 4. According to the present example, air between the shooting member 4 and the impact ball 6 can escape to the outside through the penetration hole 105c. Therefore, the impact ball 6 is prevented from being pressed by compressed air and coming off from the holder 105, and thus shooting of the impact ball 6 can be limited to shooting by means of a collision of the shooting member 4. From this, according to the present example, the impact ball 6 can be shot at a predetermined stable speed.

### Example 3

(Structure of Holder 205)

Example 3 shows an et example of the structure of the end portion of the holder 5 on one side in the axial direction in FIG. 1. The present example is the same as Example 1 except for the structure of an end portion of a holder 205 on one side in the axial direction, and thus detailed description will be omitted.

FIG. 10 is an enlarged side cross-sectional view showing a part in the vicinity of the end portion of the holder 205 corresponding to the holder 5 in FIG. 1 on one side in the axial direction in Example 3.

FIG. 11 is a cross-sectional view of the holder 205 along XI-XI in FIG. 10.

The restitution coefficient measuring device 1 of the present example has an elastic member 220 which is a member independent from the holder 205. The elastic member 220 is made of an elastic material such as a rubber or a metal, for example, and the material thereof does not matter particularly. The elastic member 220 is an annular member. In the present example, the elastic member 220 has a seamlessly annular O-shape, but the elastic member 220 may have a disconnected annular C-shape. In the case of a C-shape, the elastic member 220 may be made of to metal, and the material thereof does not matter particularly. The shape of the elastic member 220 viewed in the axial direction is an elliptical shape in which the length in the vertical direction is shorter than that in the lateral direction in FIG. 11. The holder 205 is a tubular member and has a tube hole 205b. The holder 205 has a hole 205e extending in the axial direction at an end portion on one side in the axial direction. An inner diameter of the hole 205e is larger than an inner diameter of the tube hole 205b. The inner diameter of the hole 205e is larger than the diameter of the impact ball 6. The inner diameter of the tube hole 205b is smaller than the diameter of the impact ball 6. The holder 205 has a tapered portion 205d gradually decreasing in size from the inner diameter of the hole 205e larger than the diameter of the impact ball 6 to the inner diameter of the tube hole 205b smaller than the diameter of the impact ball 6 from one side in the axial direction toward the other side in the axial direction. The impact ball 6 inserted from one side of the holder 205 in the axial direction comes into contact with the tapered portion 205d, thereby being subjected to positioning. The tapered portion 205d is an example of a positioning portion performing positioning of the impact ball 6. A positioning portion other than the tapered portion 205d may be configured to protrude in the hole 205e to a side inward in the radial direction.

The holder 205 has penetration holes 205f1 and 205f2 penetrating the holder 205 from a side outward in the radial direction to a side inward in the radial direction in the hole 205e. Each of the penetration holes 205f1 and 205f2 extends in the radial direction. The penetration hole 205f1 is disposed on the upper side in FIGS. 10 and 11, and the penetration hole 205f2 is disposed on the lower side in FIGS. 10 and 11. The elastic member 220 is fitted from an outer circumferential side of the holder 205. An inner circumference of the elastic member 220 is fitted into the penetration holes 205f1 and 205f2. The inner circumference of the elastic member 220 fitted into the penetration holes 205f1 and 205f2 protrudes to a side inward in the radial direction beyond an inner circumference of the hole 205e. A distance between the inner circumference of the elastic member 220 protruding from the penetration hole 205f1 to a side inward in the radial direction and the inner circumference of the elastic member 220 protruding from the penetration hole 205f2 to a side inward in the radial direction is smaller than the diameter of the impact ball 6, and the impact ball 6 is held by the elastic member 220. The impact ball 6 comes into contact with the elastic member 220 at two places, that is, an upper portion and a lower portion of the inner circumference of the elastic member 220. The present invention is not limited thereto, and the impact ball 6 may come into contact with the elastic member 220 at least at one or more places in the inner circumference of the elastic member 220. According to the present example, the holding position and a holding force of the impact ball 6 can be managed by the dimensions of the elastic member 220. In addition, the tapered portion 205d is used as a stopper of the impact ball 6 so that the holding position of the impact ball 6 and the shooting distance of the impact ball 6 can be made uniform with accuracy every time a shooting operation is performed. In addition, when a holding force for holding the impact ball 6 becomes weak, the holding force for holding the impact ball 6 can be recovered simply by replacing the elastic member 220. In addition, since the elastic member 220 is on a side outward in the radial direction from the holder 205, assembly and replacement are facilitated.

The holder 205 has a penetration hole 205c penetrating the holder 205 from a side outward in the radial direction to a side inward in the radial direction on the other side in the axial direction from the impact ball 6 and one side in the axial direction from the shooting member 4. According to the present example, air between the shooting member 4 and the impact ball 6 can escape to the outside through the penetration hole 205c. Therefore, the impact ball 6 is prevented from being pressed by compressed air and coming off from the holder 205, and thus shooting of the impact ball 6 can be limited to shooting by means of a collision of the shooting member 4. From this, according to the present example, stable shooting of the impact ball 6 can be performed.

### Example 4

(Structure of Holder 305)

Example 4 shows an et example of the structure of the end portion of the holder 5 on one side in the axial direction in FIG. 1. The present example is the same as Example 1 except for the structure of an end portion of a holder 305 on one side in the axial direction, and thus detailed description will be omitted.

FIG. 12 is an enlarged side cross-sectional view showing a part in the vicinity of the end portion of the holder 305 corresponding to the holder 5 in FIG. 1 on one side in the axial direction in Example 4.

FIG. 13 is a cross-sectional view of the holder 305 along XIII-XIII in FIG. 12.

The restitution coefficient measuring device 1 of the present example has an elastic member 320 which is a member independent from the holder 305. The elastic member 320 is made of an elastic material such as a rubber or a metal, for example, and the material thereof does not matter particularly. The elastic member 320 is an annular member. In the present example, the elastic member 320 is a toric member. In the present example, the elastic member 320 has a seamlessly annular O-shape, but the elastic member 320 may have a disconnected annular C-shape. In addition, as shown in FIG. 13, a cross-sectional shape of the elastic member 320 in a direction orthogonal to the axial direction is a shape having protruding portions 320b protruding from an outer circumferential portion 320a to a side inward in the radial direction. In the present example, the elastic member 320 has a shape having the protruding portions 320b at four places at equal intervals in the circumferential direction. The present invention is not limited thereto, and the elastic member 320 may have a shape having the protruding portions 320b at least at one or more places in the circumferential direction. The holder 305 is a tubular member and has a tube hole 305b. The holder 305 has a hole 305e extending in the axial direction at an end portion on one side in the axial direction. An inner diameter of the hole 305e is larger than an inner diameter of the tube hole 305b. The inner diameter of the hole 305e is larger than the diameter of the impact ball 6. The inner diameter of the tube hole 305b is smaller than the diameter of the impact ball 6. The holder 305 has a tapered portion 305d gradually decreasing in size from the inner diameter of the hole 305e larger than the diameter of the impact ball 6 to the inner diameter of the tube hole 305b smaller than the diameter of the impact ball 6 from one side in the axial direction toward the other side in the axial direction. The impact ball 6 inserted from one side of the holder 305 in the axial direction comes into contact with the tapered portion 305d, thereby being subjected to positioning. The tapered portion 305d is an example of a positioning portion performing positioning of the impact ball 6. A positioning portion other than the tapered portion 305d may be configured to protrude in the hole 305e to a side inward in the radial direction.

The holder 305 has a groove portion 305f recessed to a side outward in the radial direction in the hole 305e. The outer circumferential portion 320a of the elastic member 320 is fitted into the groove portion 305f. End portions of the protruding portions 320b of the elastic member 320 on a side inward in the radial direction fitted into the groove portion 305f are positioned on a side inward in the radial direction from the position of an outer circumferential surface of the impact ball 6 in the radial direction, and the impact ball 6 is held by the protruding portions 320b of the elastic member 320. The impact ball 6 comes into contact with the elastic member 320 at the positions of the protruding portions 320b of the elastic member 320 in the circumferential direction. According to the present example, the holding position and a holding force of the impact ball 6 can be managed by the dimensions of the elastic member 320. In addition, the tapered portion 305d is used as a stopper of the impact ball 6 so that the holding position of the impact ball 6 and the shooting distance of the impact ball 6 can be made uniform every time a shooting operation is performed. In addition, when a holding force for holding the impact ball 6 becomes weak, the holding force for holding the impact ball 6 can be recovered simply by replacing the elastic member 320.

The holder 305 has a penetration hole 305c penetrating the holder 305 from a side outward in the radial direction to a side inward in the radial direction on the other side in the axial direction from the impact ball 6 and one side in the axial direction from the shooting member 4. According to the present example, air between the shooting member 4 and the impact ball 6 can escape to the outside through the penetration hole 305c. Therefore, the impact ball 6 is prevented from being pressed by compressed air and coming off from the holder 305, and thus shooting of the impact ball 6 can be limited to shooting by means of a collision of the shooting member 4. From this, according to the present example, the impact ball 6 can be shot t a predetermined stable speed.

### Example 5

(Structure of Holder 405)

Example 5 shows another example of the structure of the end portion of the holder 5 on one side in the axial direction in FIG. 1. The present example is the same as Example 1 except for the structure of an end portion of a holder 405 on one side in the axial direction, and thus detailed description will be omitted.

FIG. 14 is an enlarged side cross-sectional view showing a part in the vicinity of the end portion of the holder 405 corresponding to the holder 5 in FIG. 1 on one side in the axial direction in Example 5.

FIG. 15 is a cross-sectional view of the holder 405 along XV-XV in FIG. 14.

FIG. 16 is a cross-sectional view showing the holder 405 along XV-XV in which the impact ball 6 and an elastic member 420 are deleted from FIG. 15.

The restitution coefficient measuring device 1 of the present example has the elastic member 420 which is a member independent from the holder 405. The elastic member 420 is made of an elastic material such as a rubber or a metal, for example, and the material thereof does not matter particularly. The elastic member 420 is an annular member. In the present example, the elastic member 420 is a toric member. In the present example, the elastic member 420 has a seamlessly annular O-shape, but the elastic member 420 may have a disconnected annular C-shape. In addition, as shown in FIG. 15 a cross-sectional shape of the elastic member 420 in a direction orthogonal to the axial direction is a shape having protruding portions 420b protruding from at outer circumferential portion 420a to a side inward in the radial direction. In the present example, the elastic member 420 has a shape having the protruding portions 420b at four places at equal intervals in the circumferential direction. The present invention is not limited thereto, and the elastic member 420 may have a shape having the protruding portions 420b at least at one or more places in the circumferential direction. The holder 405 is a tubular member and has a tube hole 405b. The holder 405 has a hole 405e extending in the axial direction at an end portion on one side in the axial direction. An inner diameter of the hole 405e is larger than an inner diameter of the tube hole 405b. The inner diameter of the hole 405e is larger than the diameter of the impact ball 6. The inner diameter of the tube hole 405b is smaller than the diameter of the impact ball 6. The holder 405 has a tapered portion 405d gradually decreasing in size from the inner diameter of the hole 405e larger than the diameter of the impact ball 6 to the inner diameter of the tube hole 405b smaller than the diameter of the impact ball 6 from one side in the axial direction toward the other side in the axial direction. The impact ball 6 inserted from one side of the holder 405 in the axial direction comes into contact with the tapered portion 405d, thereby being subjected to positioning. The tapered portion 405d is an example of a positioning portion performing positioning of the impact ball 6. A positioning portion other than the tapered portion 405d may be configured to protrude in the hole 405e to a side inward in the radial direction.

The holder 405 has penetration holes 405f1, 405f2, 405f3, and 405f4 penetrating the holder 405 from a side outward in the radial direction to a side inward in the radial direction in the hole 405e. The penetration hole 405f1 is disposed on the upper side in FIGS. 14, 15, and 16, the penetration hole 405f2 is disposed on the lower side in FIGS. 14, 15, and 16, the penetration hole 405f3 is disposed on the right side in FIGS. 15 and 16, and the penetration hole 405f4 is disposed on the left side in FIGS. 15 and 16. The elastic member 420 is fitted from an outer circumferential side of the holder 405. The protruding portions 420b of the elastic member 420 are fitted into the penetration holes 405f1, 405f2, 405f3, and 405f4. End portions of the protruding portions 420b of the elastic member 420 on a side inward in the radial direction fitted into the penetration holes 405f1, 405f2, 405f3, and 405f4 protrude to a side inward in the radial direction beyond an inner circumference of the hole 405e. A distance between the end portion of the protruding portion 420b on a side inward in the radial direction protruding from the penetration hole 405f1 to a side inward in the radial direction and the end portion of the protruding portions 420b on a side inward in the radial direction protruding from the penetration hole 405f2 to a side inward in the radial direction is smaller than the diameter of the impact ball 6. A distance between the end portion of the protruding portions 420b on a side inward in the radial direction protruding from the penetration hole 405f3 to a side inward in the radial direction and the end portion of the protruding portions 420b on a side inward in the radial direction protruding from the penetration hole 405f4 to a side inward in the radial direction is smaller than the diameter of the impact ball 6. Accordingly, the impact ball 6 is held by the elastic member 420. The impact ball 6 comes into contact with the elastic member 420 at four places, that is, an upper portion, a lower portion, a right portion, and a left portion of an inner circumference of the elastic member 420. The present invention is not limited thereto, and the impact ball 6 may come into contact with the elastic member 420 at least at one or more places in the inner circumference of the elastic member 420. According to the present example, the holding position and a holding force of the impact ball 6 can be managed by the dimensions of the elastic member 420. In addition, the tapered portion 405d is used as a stopper of the impact ball 6 so that the holding position of the impact ball 6 and the shooting distance of the impact ball 6 can be made uniform every time a shooting operation is performed. In addition, when a holding force for holding the impact ball 6 becomes weak, the holding force for holding the impact ball 6 can be recovered simply by replacing the elastic member 420. In addition, since the elastic member 420 is on a side outward in the radial direction fruit the holder 405, assembly and replacement are facilitated.

The holder 405 has a penetration hole 405c penetrating the holder 405 from a side outward in the radial direction to a side inward in the radial direction on the other side in the axial direction from the impact ball 6 and one side in the axial direction from the shooting member 4. According to the present example, air between the shooting member 4 and the impact ball 6 can escape to the outside through the penetration hole 405c. Therefore, the impact ball 6 is prevented from being pressed by compressed air and coming off from the holder 405, and thus shooting of the impact ball 6 can be limited to shooting by means of a collision of the shooting member 4. From this, according to the present example, stable shooting of the impact ball 6 can be performed.

### Example 6

(Structure of Holder 505)

Example 6 shows another example of the structure of the end portion of the holder 5 on one side in the axial direction in FIG. 1. The present example is the same as Example 1 except for the structure of an end portion of a holder 505 on one side in the axial direction, and thus detailed description will be omitted.

FIG. 17 is an enlarged side cross-sectional view showing a part in the vicinity of the end portion of the holder 505 corresponding to the holder 5 in FIG. 1 on one side in the axial direction in Example 6.

FIG. 18 is a cross-sectional view of the holder 505 along in FIG. 17.

FIG. 19 is a plan view of the holder 505 in FIG. 17 viewed from above.

The restitution coefficient measuring device 1 of the present example has elastic members 520-1 and 520-2 which are members independent from the holder 505. Each of the elastic members 520-1 and 520-2 is an elastic leaf spring. The elastic member 520-1 and the elastic member 520-2 are members having the same shape. The elastic member 520-1 extends from an end portion on the other side in the axial direction to one side in the axial direction, is bent to a side outward in the radial direction at a bent portion 520-1a, is bent to a side inward in the radial direction at a bent portion 520-1b and is bent to a side outward in the radial direction at a bent portion 520-1c. The elastic member 520-2 extends from an end portion on the other side in the axial direction to one side in the axial direction, is bent to a side outward in the radial direction at a bent portion 520-2a, is bent to a side inward in the radial direction at a bent portion 520-2b, and is bent to a side outward in the radial direction at a bent portion 520-2c. The holder 505 is a tubular member and has a tube hole 505b. The holder 505 has a hole 505e extending in the axial direction at an end portion on one side in the axial direction. An inner diameter of the hole 505e is larger than an inner diameter of the tube hole 505b. The inner diameter of the hole 505e is larger than the diameter of the impact ball 6. The inner diameter of the tube hole 505b is smaller than the diameter of the impact ball 6. The holder 505 has a tapered portion 505d gradually decreasing in size from the inner diameter of the hole 505e larger than the diameter of the impact ball 6 to the inner diameter of the tube hole 505b smaller than the diameter of the impact ball 6 from one side in the axial direction toward the other side in the axial direction. The impact ball 6 inserted from one side of the holder 505 in the axial direction comes into contact with the tapered portion 505d, thereby being subjected to positioning. The tapered portion 505d is an example of a positioning portion per positioning of the impact ball 6. A positioning portion other than the tapered portion 505d may be configured to protrude in the hole 505e to a side inward in the radial direction.

The holder 505 has a groove portion 505g1 into which an end portion of the elastic member 520-1 on the other side in the axial direction is fitted in the outer circumference. The groove portion 505g1 is a groove portion recessed to a side inward in the radial direction from an outer circumferential surface of the holder 505. In the present example, the bolder 505 has the groove portion 505g1 on the upper side in FIG. 17. The end portion of the elastic member 520-1 on the other side in the axial direction abuts a stepped portion of an end portion of the groove portion 505g1 on the other side in the axial direction. The end portion of the elastic member 520-1 on the other side in the axial direction is fixed to a bottom portion of the groove portion 505g1 by performing screwing, welding, or the like.

The holder 505 has a groove portion 505g2 into which an end portion of the elastic member 520-2 on the other side in the axial direction is fitted in the outer circumference. The groove portion 505g2 is a groove portion recessed to a side inward in the radial direction from the outer circumferential surface of the holder 505. In the present example, the holder 505 has the groove portion 505g2 on the lower side in FIG. 17. The end portion of the elastic member 520-2 on the other side in the axial direction abuts a stepped portion of an end portion of the groove portion 505g2 on the other side in the axial direction. The end portion of the elastic member 520-2 on the other side in the axial direction is fixed to a bottom portion of the groove portion 505g2 by performing screwing, welding, or the like.

The holder 505 has penetration holes 505f1 and 505f2 penetrating the holder 505 from a side outward in the radial direction to a side inward in the radial direction in the hole 505e. The penetration hole 505f1 is disposed on the upper side in FIGS. 17 and 18, and the penetration hole 505f2 is disposed on the lower side in FIGS. 17 and 18. The end portion of the elastic member 520-1 on the other side in the axial direction is fixed to the groove portion 505g1 from the outer circumferential side of the holder 505. The end portion of the elastic member 520-1 on one side in the axial direction is not fixed. The elastic member 520-1 is fitted into the penetration hole 505f1 by means of the elasticity of the elastic member 520-1, and the bent portion 520-1c protrudes to a side inward in the radial direction beyond an inner circumferential surface of the hole 505e. The end portion of the elastic member 520-2 on the other side in the axial direction is fixed to the groove portion 505g2 from the outer circumferential side of the holder 505. The end portion of the elastic member 520-2 on one side in the axial direction is not fixed. The elastic member 520-2 is fitted into the penetration hole 505f2 by means of the elasticity of the elastic member 520-2, and the hem portion 520-2c protrudes to a side inward in the radial direction beyond the inner circumferential surface of the hole 505e.

A distance between the bent portion 520-1c of the elastic member 520-1 protruding from the penetration hole 505f1 to a side inward in the radial direction and the bent portion 520-2c of the elastic member 520-2 protruding from the penetration hole 505f2 to a side inward in the radial direction is smaller than the diameter of the impact ball 6, and the impact ball 6 is held by the elastic member 520-1 and the elastic member 520-2. The impact ball 6 comes into contact with the elastic members 520-1 and 520-2 at two places, that is, an upper portion and a lower portion of an inner circumference of the holder 505. The present invention is not limited thereto, and the impact ball 6 may come into contact with the elastic members at least at one or more places in the inner circumference of the holder 505. In this case, it is favorable to provide as many elastic members (leaf springs) as the number of contact places. According to the present example, the holding position and a holding force of the impact ball 6 can be managed by the dimensions of the elastic members 520-1 and 520-2. In addition, the tapered portion 505d is used as a stopper of the impact ball 6 so that the holding position of the impact ball 6 and the shooting distance of the impact ball 6 can be made uniform every time a shooting operation is performed. In addition, when a holding three for holding the impact ball 6 becomes weak, the holding force for bolding the impact ball 6 can be recovered simply by replacing the elastic members 520-1 and 520-2. In addition, since the elastic members 520 are on a side outward in the radial direction from the holder 205, assembly and replacement are facilitated.

The holder 505 has a penetration hole 505c penetrating the holder 505 from a side outward in the radial direction to a side inward in the radial direction on the other side in the axial direction from the impact ball 6 and one side in the axial direction from the shooting member 4. According to the present example, air between the shooting member 4 and the impact ball 6 can escape to the outside through the penetration hole 505c. Therefore, the impact ball 6 is prevented from being pressed by compressed air and coming off from the holder 505, and thus shooting of the impact ball 6 can be limited to shooting by means of a collision of the hooting member 4. From this, according to the present example, the impact ball 6 can be shot at a predetermined stable shooting speed.

### Example 7

(Structure of Holder 605)

Example 7 shows another example of the structure of the end portion of the holder 5 on one side in the axial direction in FIG. 1. The present example is the same as Example 1 except for the structure of an end portion of a holder 605 on one side in the axial direction, and thus detailed description will be omitted.

FIG. 20 is an enlarged side cross-sectional view showing a part in the vicinity of the end portion of the holder 605 corresponding to the holder 5 in FIG. 1 on one side in the axial direction in Example 7.

FIG. 21 is a cross-sectional view of the holder 605 along XXI-XXI in FIG. 20.

The restitution coefficient measuring device 1 of the present example has elastic members 620 which are members independent from the holder 605, and push-pins 630-1 and 630-2. The elastic members 620 are made of an elastic material such as a rubber or a metal, for example, and the material thereof does not matter particularly. The elastic members 620 are annular members. The elastic members 620 perform energizing to a side inward in the radial direction. In the present example, the elastic members 620 are toric members. In the present example, the elastic members 620 have a disconnected annular C-shape as shown in FIG. 21, but the elastic members 620 may have a seamlessly annular O-shape. The push-pin 630-1 and the push-pin 630-2 are members having the same shape. The push-pin 630-1 has a head portion 630-1a having a larger diameter than a penetration hole 605f1, and an insertion portion 630-1b having a smaller diameter than the penetration hole 605f1. The head portion 630-1a has a groove portion 630-1c extending in the circumferential direction and recessed to a side inward in the radial direction on a side outward in the radial direction. A length of the insertion portion 630-1b in the radial direction is longer than a depth of the penetration hole 605f1. The push-pin 630-2 has a head portion 630-2a having a larger diameter than a penetration hole 605f2, and an insertion portion 630-2b having a smaller diameter than the penetration hole 605f2. The head portion 630-2a has a groove portion 630-2c extending in the circumferential direction and recessed to a side inward in the radial direction on a side outward in the radial direction. A length of the insertion portion 630-2b in the radial direction is longer than a depth of the penetration hole 605f2.

The holder 605 is a tubular member and has a tube hole 605b. The holder 605 has a hole 605e extending in the axial direction at an end portion on one side in the axial direction. An inner diameter of the hole 605e is larger than an inner diameter of the tube hole 605b. The inner diameter of the hole 605e is larger than the diameter of the impact ball 6. The inner diameter of the tribe hole 605b is smaller than the diameter of the impact ball 6. The holder 605 has a tapered portion 605d gradually decreasing in size from the inner diameter of the hole 605e larger than the diameter of the impact ball 6 to the inner diameter of the tube hole 605b smaller than the diameter of the impact ball 6 from one side in the axial direction toward the other side in the axial direction. The impact ball 6 inserted from one side of the holder 605 in the axial direction comes into contact with the tapered portion 605d, thereby being subjected to positioning. The tapered portion 605d is an example of a positioning portion performing positioning of the impact ball 6. A positioning portion other than the tapered portion 605d may be configured to protrude in the hole 605e to a side inward in the radial direction.

The holder 605 has the penetration holes 605f1 and 605f2 penetrating the holder 605 from a side outward in the radial direction to a side inward in the radial direction in the hole 605e. The penetration hole 605f1 is disposed on the upper side in FIGS. 20 and 21, and the penetration hole 605f2 is disposed on the lower side in FIG. 20 and 21. The insertion portion 630-1b of the push-pin 630-1 is fitted into the penetration hole 605f1. The insertion portion 630-2b of the push-pin 630-2 is fitted into the penetration hole 605f2. In this state, the elastic members 620 are fitted into the groove portion 630-1c of the head portion 630-1a of the push-pin 630-1 and the groove portion 630-2c of the head portion 630-2a of the push-pin 630-2. The push-pins 630-1 and 630-2 and the elastic members 620 function as plungers. An end portion of the insertion portion 630-1b on a side inward in the radial direction protrudes to a side inward in the radial direction beyond an inner circumferential surface of the hole 605e.

A distance between the insertion portion 630-1b protruding from the penetration hole 605f1 to a side inward in the radial direction and the insertion portion 630-2b protruding from the penetration hole 605f2 to a side inward in the radial direction is smaller than the diameter of the impact ball 6, and the impact ball 6 is held by the push-pins 630-1 and 630-2 which are biased by the elastic members 620. The impact ball 6 comes into contact with the push-pins 630-1 and 630-2 at two places, that is, an upper portion and a lower portion of an inner circumference of the holder 605. The present invention is not limited thereto, and the impact ball 6 may come into contact with the push-pins at least at one or more places in the inner circumference of the holder 605. In this case, it is favorable to provide as many push-pins as the number of contact places. According to the present example, the holding position and a holding force of the impact ball 6 can be managed by the dimensions of the elastic members 620 and the push-pins 630-1 and 630-2. In addition, the tapered portion 605d is used as a stopper of the impact ball 6 so that the holding position of the impact ball 6 and the shooting distance of the impact ball 6 can be made uniform every time a shooting operation is performed. In addition, when a holding force for holding the impact ball 6 becomes weak, the holding force for holding the impact ball 6 can be recovered simply by replacing the elastic members 620 and the push-pins 630-1 and 630-2. In addition, since the elastic members 620 and the push-pins 630-1 and 630-2 are on a side outward in the radial direction from the holder 605, assembly and replacement are facilitated.

The holder 605 has a penetration hole 605c. penetrating the holder 605 from a side outward in the radial direction to a side inward in the radial direction on the other side in the axial direction from the impact ball 6 and one side in the axial direction from the shooting member 4. According to the present example, air between the shooting member 4 and the impact ball 6 can escape to the outside through the penetration hole 605c. Therefore, the impact ball 6 is prevented from being pressed by compressed air and coming off from the holder 605, and thus shooting of the impact ball 6 can be limited to shooting by means of a collision of the shooting member 4. From this, according to the present example, the impact ball 6 can be shot at a predetermined stable speed.

### Example 8

(Structure of Holder 705)

Example 8 shows another example of the structure of the end portion of the holder 5 on one side in the axial direction in FIG. 1. The present example is the same as Example 1 except for the structure of an end portion of a holder 705 on one side in the axial direction, and thus detailed description will be omitted.

FIG. 22 is an enlarged side cross-sectional view showing a part in the vicinity of the end portion of the holder 705 corresponding to the holder 5 in FIG. 1 on one side in the axial direction in Example 8.

FIG. 23 is a cross-sectional view of the holder 705 along XXIII-XXIIII in FIG. 22.

The restitution coefficient measuring device 1 of the present example has elastic members 720-1 and 720-2 which are members independent from the holder 705, and push-pins 730-1 and 730-2. Each of the elastic members 720-1 and 720-2 is an elastic leaf spring.

The elastic member 720-1 and the elastic member 720-2 are members having the same shape. The elastic member 720-1 extends from an end portion on the other side in the axial direction to one side in the axial direction, is bent to a side outward in the radial direction at a bent portion 720-1a, and is bent to a side inward in the radial direction at a bent portion 720-1b. The elastic member 720-2 extends from an end portion on the other side in the axial direction to one side in the axial direction, is bent to a side outward in the radial direction at a bent portion 720-2a, and is bent to a side inward in the radial direction at a bent portion 720-2b.

The push-pin 730-1 and the push-pin 730-2 are members having the same shape. The push-pin 730-1 has a head portion 730-1a having a larger diameter than a penetration hole 705f1, and an insertion portion 730-1b having a smaller diameter than the penetration hole 705f1. A length of the insertion portion 730-1b in the radial direction is longer than a depth of the penetration hole 705f1. The push-pin 730-2 has a head portion 730-2a haying a larger diameter than a penetration hole 705f2, and an insertion portion 730-2b having a smaller diameter than the penetration hole 705f2. A length of the insertion portion 730-2b in the radial direction is longer than a depth of the penetration hole 705f2.

The holder 705 is a tubular member and has a tube hole 705b. The holder 705 has a hole 705e extending in the axial direction at an end portion on one side in the axial direction. An inner diameter of the hole 705e is larger than an inner diameter of the tube hole 705b. The inner diameter of the hole 705e is larger than the diameter of the impact ball 6. The inner diameter of the tube hole 705b is smaller than the diameter of the impact ball 6. The holder 705 has a tapered portion 705d gradually decreasing in size from the inner diameter of the hole 705e larger than the diameter of the impact ball 6 to the inner diameter of the tube hole 705b smaller than the diameter of the impact ball 6 from one side in the axial direction toward the other side in the axial direction. The impact ball 6 inserted from one side of the holder 705 in the axial direction comes into contact with the tapered portion 705d, thereby being subjected to positioning. The tapered portion 705d is an example of a positioning portion performing positioning of the impact ball 6. A positioning portion other than the tapered portion 705d may be configured to protrude in the hole 705e to a side inward in the radial direction.

The holder 705 has a groove portion 705g1 into which an end portion of the elastic member 720-1 on the other side in the axial direction is fitted in the outer circumference. The groove portion 705g1 is a groove portion recessed to a side inward in the radial direction from an outer circumferential surface of the holder 705. In the present example, the holder 705 has the groove portion 705g1 on the upper side in FIG. 22. The end portion of the elastic member 720-1 on the on the other side in the axial direction abuts a stepped portion of an end portion of the groove portion 705g1 on the other side in the axial direction. The end portion of the elastic member 720-1 on the other side in the axial direction is fixed to a bottom portion of the groove portion 705g1 by performing screwing, welding, or the like.

The holder 705 has a groove portion 705g2 into which an end portion of the elastic member 720-2 on the other side in the axial direction is fitted in the outer circumference. The groove portion 705g2 is a groove portion recessed to a side inward in the radial direction from the outer circumferential surface of the holder 705. In the present example, the holder 705 has the groove portion 705g2 on the lower side in FIG. 22. The end portion of the elastic member 720-2 on the other side in the axial direction abuts a stepped portion of an end portion of the groove portion 705g2 on the other side in the axial direction. The end portion of the elastic member 720-2 on the other side in the axial direction is fixed to a bottom portion of the groove portion 705g2 by performing screwing, welding, or the like.

The holder 705 has penetration holes 705f1 and 705f2 penetrating the holder 705 from a side outward in the radial direction to a side inward in the radial direction in the hole 705e. The penetration hole 705f1 is disposed on the upper side in FIGS. 22 and 23, and the penetration hole 705f2 is disposed on the lower side in FIGS. 22 and 23. The insertion portion 730-1b of the push-pin 730-1 is fitted into the penetration hole 705f1. The insertion portion 730-2b of the push-pin 730-2 is fitted into the penetration hole 705f2. In this state, the end portion of the elastic member 720-1 on one side in the axial direction is fixed to the head portion 730-1a of the push-pin 730-1 on a side outward in the radial direction by a fixing member 730-1c such as a screw, and the end portion of the elastic member 720-2 on one side in the axial direction is fixed to the head portion 730-2a of the push-pin 730-2 on a side outward in the radial direction by a fixing member 730-2c such as a screw The push-pins 730-1 and 730-2 and the elastic members 720-1 and 720-2 function as plungers. An end portion of the insertion portion 730-1b on a side inward in the radial direction protrudes to a side inward in the radial direction beyond an inner circumferential surface of the hole 705e.

A distance between the insertion portion 730-1b protruding from the penetration hole 705f1 to a side inward in the radial direction and the insertion portion 730-2b protruding from the penetration hole 705f2 to a side inward in the radial direction is smaller than the diameter of the impact ball 6, and the impact ball 6 is held by the push-pins 730-1 and 730-2 which are biased by the elastic members 720-1 and 720-2. The impact ball 6 comes into contact with the push-pins 730-1 and 730-2 at two places, that is, an upper portion and a lower portion of an inner circumference of the holder 705. The present invention is not limited thereto, and the impact ball 6 may come into contact with the push-pins at least at one or more places in the inner circumference of the holder 705. In this case, it is favorable to provide as many push-pins as the number of contact places. According to the present example, the holding position and a holding force of the impact ball 6 can be managed by the dimensions of the elastic members 720-1 and 720-2 and the push pins 730-1 and 730-2. In addition, the tapered portion 705d is used as a stopper of the impact ball 6 so that the holding position of the impact ball 6 and the shooting distance of the impact ball 6 can be made uniform every time a shooting operation is performed. In addition, when a holding force for holding the impact ball 6 becomes weak, the holding force for holding the impact ball 6 can be recovered simply by replacing the elastic members 720-1 and 720-2 and the push-pins 730-1 and 730-2. In addition, since the elastic members 720-1 and 720-2 and the push-pins 730-1 and 730-2 are on a side outward in the radial direction from the holder 705, assembly and replacement are facilitated.

The holder 705 has a penetration hole 705c penetrating the holder 705 from a side outward in the radial direction to a side inward in the radial direction on the other side in the axial direction from the impact ball 6 and one side in the axial direction from the shooting member 4. According to the present example, air between the shooting member 4 and the impact ball 6 can escape to the outside through the penetration hole 705c. Therefore, the impact ball 6 is prevented from being pressed by compressed air and coming off from the bolder 705, and thus shooting of the impact ball 6 can be limited to shooting by means of a collision of the shooting member 4. From this, according to the present example, the impact ball 6 can be shot at a predetermined stable speed.

### Example 9

(Structure of Holder 805)

Example 9 shows another example of the structure of the end portion of the holder 5 on one side in the axial direction in FIG. 1. In the present example, is the same as Example 1 except for the structure of an end portion of a holder 805 on one side in the axial direction, and thus detailed description will be omitted.

FIG. 24 is an enlarged side cross-sectional view showing a part in the vicinity of the end portion of the holder 805 corresponding to the holder 5 in FIG. 1 on one side in the axial direction in Example 9.

FIG. 25 is a cross-sectional view of the holder 805 along XXV-XXV in FIG. 24.

The restitution coefficient measuring device 1 of the present example has an elastic member 820 which is a member independent from the holder 805. The holder 805 is a tubular member and has a tube hole 805b. The elastic member 820 is made of an elastic material such as a rubber or a metal, for example, and the material thereof does not matter particularly. The elastic member 820 is an annular member. In the present example, the elastic member 820 is a cylindrical member. In the present example, the elastic member 820 has a seamlessly annular O-shape, but the elastic member 820 may have a disconnected annular C-shape. The elastic member 820 has a tube hole 820b extending in the axial direction at an end portion on the other side in the axial direction. The elastic member 820 has a hole 820c extending in the axial direction at an end portion on one side in the axial direction. An inner diameter of the hole 820c is larger than an inner diameter of the tube hole 820b. The inner diameter of the hole 820c is smaller than the diameter of the impact ball 6. The inner diameter of the hole 820c has a size to an extent that the impact ball 6 can be held. The inner diameter of the tube hole 820b is smaller than the diameter of the impact ball 6. The inner diameter of the tube hole 820b has a size to an extent that the impact ball 6 cannot be inserted. The elastic member 820 has a tapered portion 820d gradually decreasing in size from the inner diameter of the hole 820c to the inner diameter of the tube hole 820b from one side in the axial direction toward the other side in the axial direction. The impact ball 6 inserted from one side of the elastic member 820 in the axial direction comes into contact with a tapered portion 805d, thereby be ng subjected to positioning. The tapered portion 805d is an example of a positioning portion performing positioning of the impact ball 6. A positioning portion other than the tapered portion 805d may be configured to protrude in the hole 820c to a side inward in the radial direction. The impact ball 6 is held in the hole 820c of the elastic member 820. The elastic member 820 has a thin wall portion 820a having a larger inner diameter than that on one side in the axial direction at an end portion on the other side in the axial direction. The holder 805 has a thin wall portion 805a having a smaller outer diameter than that on the other side in the axial direction at an end portion on one side in the axial direction. The thin wall portion 820a of the elastic member 820 is fitted to the thin wall portion 805a of the holder 805 on a side outward in the radial direction. According to the present example, the impact ball 6 can be held by means of only the elasticity of the material of the elastic member 820. In addition, the tapered portion 805d is used as a stopper of the impact ball 6 so that the holding position of the impact ball 6 and the shooting distance of the impact ball 6 can be made uniform every time a shooting operation is performed. When a holding force for holding the impact ball 6 becomes weak, the holding force for holding the impact ball 6 can be recovered simply by replacing the elastic member 820.

The holder 805 has a penetration hole 805c penetrating the holder 805 from a side outward in the radial direction to a side inward in the radial direction on the other side in the axial direction from the impact ball 6 and one side in the axial direction from the shooting member 4. When the shooting member 4 moves to one side in the axial direction, air between the shooting member 4 and the impact ball 6 is compressed. For this reason, when the penetration hole 805c is not provided, there is a possibility that the impact ball 6 may be pressed by compressed air and come off from the holder 5. According to the present example, air between the shooting member 4 and the impact ball 6 can escape to the outside through the penetration hole 805c. Therefore, the impact ball 6 is prevented from being pressed by compressed air and coming off from the holder 5, and thus shooting of the impact ball 6 can be limited to shooting by means of a collision of the shooting member 4. From this according to the present example, the impact ball 6 can be shot at a predetermined stable speed.

In the present invention, the restitution coefficient measuring device 1 according to the examples described above can be used as a hardness measuring device. That is, the hardness measuring device includes the holder 5 that holds the spherical impact ball 6, the shooting mechanism 3 for shooting the impact ball 6 held by the holder 5 from the holder 5 toward the specimen 8, the speed measuring unit that measures the collision speed which is a speed of the impact ball 6 before the impact ball 6 collides with the specimen 8 and the restitution speed which is a speed of the impact ball after the impact ball has bounced from the specimen, the computing unit 10 that calculates the hardness of the specimen 8 on the basis of a ratio of the restitution speed to the collision speed, and the elastic member 20. The elastic member 20 is a member independent from the holder 5 and is disposed on one side of the holder 5 in the axial direction, and the holder 5 holds the impact ball 6 by means of a energizing force of the elastic member 20. For example, the computing unit 10 calculates the hardness of the specimen 8 by multiplying the ratio of the restitution speed of the impact ball 6 to the collision speed of the impact ball 6 by a predetermined constant of proportionality.

### Other Embodiments

The present invention is not limited to the foregoing examples and includes various modification examples. For example, the foregoing examples have been described in detail in order to facilitate the understanding of the description of the present invention, and the present invention is not necessarily limited to an embodiment including all the configurations described above. In addition, a part of the configuration of a certain example can be replaced with the configuration of a different example. In addition, the configuration of a different example can be added to the configuration of a certain example. In addition, a part of the configuration of each example can be subjected to addition, deletion, and replacement of a different configuration.

### Overview of Embodiments

In order to solve the foregoing problems, according to an embodiment of the present invention, there is provided a restitution coefficient measuring device measuring a restitution coefficient of a measuring object. The restitution coefficient measuring device includes a holder that holds a spherical impact ball colliding with the measuring object with an elastic member, a shooting mechanism for shooting the impact ball held by the holder from the holder toward the measuring object, a speed measuring unit that measures both a collision speed at which the impact ball collides with the measuring object and a restitution speed at which the impact ball bounces from the measuring object, and a computing unit that calculates the restitution coefficient on the basis of the restitution speed with respect to the collision speed. A hole for bleeding air is bored on any side surface of the holder. The elastic member is an independent member replaceable with respect to the holder and is disposed at an end portion of the holder in an axial direction.

According to the present embodiment, it is possible to provide a restitution coefficient measuring device in which stable shooting of an impact ball is improved, and a hardness measuring device.

That is, according to the present embodiment, since the elastic member is a member independent from the holder, there is no need to adjust the holder itself. A holding force for holding the impact ball can be easily adjusted by the dimensions of the elastic member which is an independent member, the position of the impact ball can be made stable, and thus more accurate measurement can be performed.

In addition, according to the present embodiment, the impact ball can be held by means of only the elasticity of the elastic member.

In addition, according to the present embodiment, since the structure for holding the impart ball is simple, the performance is stable, and the manufacturing cost can be kept low.

In addition according to the present embodiment, when a holding force becomes weak, the elastic member may be replaced, and thus the number of replacement components is reduced.

In addition, according to the present embodiment, since a hole for bleeding air is bored, the impact ball can be prevented from erroneously popping out due to air compressed by the shooting mechanism.

In addition, according to the embodiment of the present invention, in the restitution coefficient measuring device, the elastic member has an annular shape and biases the impact ball to a side inward in a radial direction.

According to the present embodiment, since the impact ball is biased to a side inward in the radial direction by the elastic member and is held thereat, the position of the impact ball can be made stable, and thus more accurate measurement can be performed.

In addition, according to the embodiment of the present invention, in the restitution coefficient measuring device, the holder has a tube portion having a tubular shape for holding the impact ball in a tube hole. The tube portion has a groove portion recessed from a side inward in the radial direction to a side outward in the radial direction throughout a whole circumference on an inner circumferential surface. The elastic member is fitted into the groove portion. At least a part of the elastic member protrudes to a side inward in the radial direction beyond the inner circumferential surface of the tube portion.

According to the present embodiment, since an outer circumferential surface of the impact ball is held in the inner circumference of the holder, the position of the impact ball can be made stable, and thus more accurate measurement can be performed.

In addition, according to the embodiment of the present invention, in the restitution coefficient measuring device, the elastic member protrudes to a side inward in the radial direction beyond the inner circumferential surface of the tube portion throughout the whole circumference.

According to the present embodiment, since the outer circumferential surface of the impact ball is held in the whole inner circumference of the holder, the position of the impact ball can be made stable, and thus more accurate measurement can be performed.

In addition, according to the embodiment of the present invention, in the restitution coefficient measuring device, the elastic member has a projection portion protruding to a side inward in the radial direction beyond the inner circumferential surface of the tube portion at least at one or more places in a circumferential direction.

According to the present embodiment, since the outer circumferential surface of the impact ball is held at a plurality of places in the inner circumference of the holder, the position of the impact ball can be made stable, and thus more accurate measurement can be performed.

According to the present embodiment, a holding force can be easily adjusted by the number of projections or a protruding amount.

In addition, according to tie embodiment of the present invention, in the restitution coefficient measuring device, the holder has a tube portion having a tubular shape for bolding the impact ball in a tube hole. The tube portion has a penetration hole penetrating the tube portion from a side outward in the radial direction to a side inward in the radial direction at a predetermined place in a circumferential direction. The elastic member is fitted on an outer circumferential surface of the tube portion and protrudes to a side inward in the radial direction beyond the inner circumferential surface of the tube portion via the penetration hole.

According to the present embodiment, since the elastic member is mounted in an outer circumference of the holder, assembly work can be facilitated. In addition, the elastic member can be easily replaced.

In addition, according to the embodiment of the preset invention in the restitution coefficient measuring device, the holder has a plurality of the penetration holes at equal intervals in the circumferential direction.

According to the present embodiment, since the outer circumferential surface of the impact ball is held at a plurality of places in the circumferential direction, the position of the impact ball can be made stable, and thus more accurate measurement can be performed.

In addition, according to the embodiment of the present invention, the restitution coefficient measuring device further includes a push-pin. The holder has a tube portion having a tubular shape for holding the impact ball in a tube hole. The tube portion has a penetration hole penetrating the tube portion from a side outward in the radial direction to a side inward in the radial direction at a predetermined place in a circumferential direction. The push-pin is fitted into the penetration hole. The elastic member is fitted at an end portion of the push-pin on a side outward in the radial direction on an outer circumferential surface of the tube portion. The push-pin protrudes to a side inward in the radial direction beyond the inner circumferential surface of the tube portion via the penetration hole.

According to the present embodiment, since the elastic member is mounted in the outer circumference of the holder, assembly work cat be facilitated. In addition, the elastic member can be easily replaced.

In addition, according to the embodiment of the present invention, in the restitution coefficient measuring device, the holder has a tube portion having a tubular shape for holding the impact ball in a tube hole. The tube portion has a penetration hole penetrating the tube portion from a side outward in the radial direction to a side inward in the radial direction at a predetermined place in a circumferential direction. The elastic member is fitted on an outer circumferential surface of the tube portion and protrudes to a side inward in the radial direction beyond the inner circumferential surface of the tube portion via the penetration hole.

According to the present embodiment, since the elastic member is mounted in the outer circumference of the holder, assembly work can be facilitated. In addition, the elastic member can be easily replaced.

In addition, according to the embodiment of the present invention, in the restitution coefficient measuring device, the elastic member is a leaf spring extending in the axial direction.

According to the present embodiment, since the outer circumferential surface of the impact ball is held by the leaf spring, the position of the impact ball can be made stable, and thus more accurate measurement can be performed.

In addition, according to the embodiment of the present invention, the restitution coefficient measuring device further includes a push-pin. The holder has a tube portion having a tubular shape for holding the impact ball in a tube hole. The tube portion has a penetration hole penetrating the tube portion from a side outward in the radial direction to a side inward in the radial direction at a predetermined place in a circumferential direction. The push-pin is fitted into the penetration hole. The elastic member biases an end portion of the push-pin on a side outward in the radial direction to a side inward in the radial direction on an outer circumferential surface of the tube portion. The push-pin protrudes to a side inward in the radial direction beyond the inner circumferential surface of the tube portion via the penetration hole.

According to the present embodiment, since the elastic member is mounted in the outer circumference of the holder, assembly work can be facilitated. In addition, the elastic member can be easily replaced.

In addition, according to the embodiment of the present invention, in the restitution coefficient measuring device, the holder has a positioning portion performing positioning of the impact ball.

According to the present embodiment, the position of the impact ball can be made stable by the positioning portion, and thus more accurate measurement can be performed.

In addition, according to the embodiment of the present invention, in the restitution coefficient measuring device, the holder has a tube portion having a tubular shape for holding the impact ball in a tube hole. The positioning portion is a tapered portion in which an inner diameter of the tube portion gradually decreases in size from a first inner diameter larger than a diameter of the impact ball to a second inner diameter smaller than the diameter of the impact ball from one side in the axial direction toward the other side in the axial direction.

According to the present embodiment, the position of the impact ball can be made stable by the tapered portion, and thus more accurate measurement can be performed.

In addition, according to another embodiment of the present invention, there is provided a restitution coefficient measuring device measuring a restitution coefficient of a measuring object. The restitution coefficient measuring device includes a holder that holds a spherical impact ball colliding with the measuring object, a shooting mechanism for shooting the impact ball held by the holder from the holder toward the measuring object, a speed measuring unit that measures both a collision speed at which the impact ball collides with the measuring object and a restitution speed at which the impact ball bounces from the measuring object, a computing unit that calculates the restitution coefficient on the basis of the restitution speed with respect to the collision speed. The holder has a tube portion having a tubular shape for holding the impact ball in a tube hole. The shooting mechanism has a shooting member disposed on the other side in an axial direction from the impact ball and capable of moving in the tube hole in the axial direction, a energizing portion applying a energizing force to the shooting member toward one side in the axial direction, and a restriction portion restricting movement of the shooting member to one side in the axial direction. The shooting member has a small diameter portion on one side in the axial direction, a large diameter portion having a larger diameter than the small diameter portion on the other side in the axial direction from the small diameter portion, and a stepped portion which is a step generated due to difference between the diameters of the small diameter portion and the large diameter portion. The restriction portion is an engagement member engaging with the stepped portion.

According to the present embodiment, the shooting member can be moved toward the impact ball by disengaging the engagement member which has engaged with the stepped portion, shooting of the impact ball can be made stable, and thus more accurate measurement can be performed.

In addition, according to the embodiment of the present invention, in the restitution coefficient measuring device, the tube portion has a penetration hole penetrating the tube portion from a side outward in a radial direction to a side inward in the radial direction on the other side in the axial direction from the impact ball and one side in the axial direction from the shooting member.

According to the present embodiment, since air pressed by the shooting member when the shooting member moves to one side in the axial direction can escape through the penetration hole, the impact ball can be prevented from being shot due to air without coming into contact with the shooting member, shooting of the impact ball can be made stable, and thus more accurate measurement can be performed.

In addition, according to another embodiment of the present invention, there is provided a hardness measuring device including a holder that holds a spherical impact ball, a shooting mechanism for shooting the impact ball held by the holder from the holder toward a specimen, a speed measuring unit that measures a collision speed which is a speed of the impact ball before the impact ball collides with the specimen and a restitution speed which is a speed of the impact ball after the impact ball has bounced from the specimen, a computing unit that calculates the hardness of the specimen on the basis of a ratio of the restitution speed to the collision speed, and an elastic member. The elastic member is a member independent from the holder and is disposed on one side of the holder in an axial direction. The holder holds the impact ball by means of a energizing force of the elastic member.

According to the present embodiment, it is possible to provide a restitution coefficient measuring device in which stable shooting of an impact ball is improved, and a hardness measuring device.

That is, according to the present embodiment, since the elastic member is a member independent from the holder, there is no need to adjust the holder itself. A holding force for holding the impact ball can be easily adjusted by the dimensions of the elastic member which is an independent member, the position of the impact ball can be made stable, and thus more accurate measurement can be performed.

In addition, according to the present embodiment, the impact ball can be held by means of only the elasticity of the elastic member.

In addition, according, to the present embodiment, when a holding force becomes weak, the elastic member may be replaced, and thus the number of replacement components is reduced.

In addition, according to another embodiment of the present invention, there is provided a hardness measuring device including a holder that holds a spherical impact ball, a shooting mechanism for shooting the impact ball held by the holder from the holder toward a specimen, a speed measuring unit that measures a collision speed which is a speed of the impact ball before the impact ball collides with the specimen and a restitution speed which is a speed of the impact ball after the impact ball has bounced from the specimen, and a computing unit that calculates the hardness of the specimen on the basis of a ratio of the restitution speed to the collision speed. The holder has a tube portion having a tubular shape for holding the impact ball in a tube hole. The shooting mechanism has a shooting member disposed on the other side in an axial direction from the impact ball and capable of moving in the tube hole in the axial direction, a energizing portion applying a energizing force to the shooting member toward one side in the axial direction, and a restriction portion restricting movement of the shooting member to one side in the axial direction. The shooting member has a small diameter portion on one side in the axial direction, a large diameter portion having a larger diameter than the small diameter portion on the other side in the axial direction from the small diameter portion, and a stepped portion which is a step generated due to a difference between the diameters of the small diameter portion and the large diameter portion. The restriction portion is an engagement member engaging with the stepped portion.

According to the present embodiment, the shooting member can be moved toward the impact ball by disengaging the engagement member which has engaged with the stepped portion, shooting of the impact ball can be made stable, and thus more accurate measurement can be performed.

What is claimed is:

1. A restitution coefficient measuring device measuring a restitution coefficient of a measuring object, the restitution coefficient measuring device comprising:
a holder that holds a spherical impact ball colliding with the measuring object with an elastic member;
a shooting mechanism for shooting the impact ball held by the holder from the holder toward the measuring object;
a speed measuring unit that measures both a collision speed at which the impact ball collides with the measuring object and a restitution speed at which the impact ball bounces from the measuring object; and
a computing unit that calculates the restitution coefficient on the basis of the restitution speed with respect to the collision speed,
wherein a hole for bleeding air is bored on any side surface of the holder, and
wherein the elastic member is an independent member replaceable with respect to the holder and is disposed at an end portion of the holder in an axial direction.

2. The restitution coefficient measuring device according to claim 1,
wherein the elastic member has an annular shape and biases the impact ball to a side inward in a radial direction.

3. The restitution coefficient measuring device according to claim 2,
wherein the holder has a tube portion having a tubular shape for holding the impact ball in a tube hole,
wherein the tube portion has a groove portion recessed from a side inward in the radial direction to a side outward in the radial direction throughout a whole circumference on an inner circumferential surface,
wherein the elastic member is fitted into the groove portion, and
wherein at least a part of the elastic member protrudes to a side inward in the radial direction beyond the inner circumferential surface of the tube portion.

4. The restitution coefficient measuring device according to claim 3,
wherein the elastic member protrudes to a side inward in the radial direction beyond the inner circumferential surface of the tube portion throughout the whole circumference.

5. The restitution coefficient measuring device according to claim 3,
wherein the elastic member has a projection portion protruding to a side inward in the radial direction beyond the inner circumferential surface of the tube portion at least at one or more places in a circumferential direction.

6. The restitution coefficient measuring device according to claim 2,
wherein the holder has a tube portion having a tubular shape for holding the impact ball in a tube hole,
wherein the tube portion has a penetration bole penetrating the tube portion from a side outward in the radial direction to a side inward in the radial direction at a predetermined place in a circumferential direction, and
wherein the elastic member is fitted on an outer circumferential surface of the tube portion and protrudes to a side inward in the radial direction beyond the inner circumferential surface of the tube portion via the penetration hole.

7. The restitution coefficient measuring device according to claim 6,
wherein the holder has a plurality of the penetration holes at equal intervals in the circumferential direction.

8. The restitution coefficient measuring device according to claim 2 further comprising:
a push-pin
wherein the holder has a tube portion having a tubular shape for holding the impact ball in a tube hole,
wherein the tube portion has a penetration hole penetrating the tube portion from a side outward it the radial direction side inward in the radial direction at a predetermined place in a circumferential direction,
wherein the push-pin is fitted into the penetration hole,
wherein the elastic member is fitted at an end portion of the push-pin on a side outward in the radial direction on an outer circumferential surface of the tube portion, and
wherein the push-pin protrudes to a side inward in the radial direction beyond the inner circumferential surface of the tube portion via the penetration hole.

9. The restitution coefficient measuring device according to claim 1,
wherein the holder has a tube portion having a tubular shape for holding the impact ball in a tube hole,
wherein tube portion has a penetration hole penetrating the tube portion from a side outward in the radial direction to a side inward in the radial direction at a predetermined place in a circumferential direction, and
wherein the elastic member is fitted on an outer circumferential surface of the tube portion and protrudes to a side inward in the radial direction beyond the inner circumferential surface of the tube portion via the penetration hole.

10. The restitution coefficient measuring device according to claim 9,
wherein the elastic member is a leaf spring extending in the axial direction.

11. The restitution coefficient measuring device according to claim 1 further comprising:
a push-pin,
wherein the holder has a tube portion having a tubular shape for holding the impact ball in a tube hole,
wherein the tube portion has a penetration hole penetrating the tube portion from a side outward in the radial direction to a side inward in the radial direction at a predetermined place in a circumferential direction,
wherein the push-pin is fitted into the penetration hole,
wherein the elastic member biases an end portion of the push-pin on a side outward in the radial direction to a side inward in the radial direction on an outer circumferential surface of the tube portion, and
wherein the push-pin protrudes to a side inward in the radial direction beyond the inner circumferential surface of the tube portion via the penetration hole.

12. The restitution coefficient measuring device according to claim 1,
wherein the holder has a positioning portion performing positioning of the impact ball.

13. The restitution coefficient measuring device according to claim 12,
wherein the holder has a tube portion having a tubular shape for holding the impact ball in a tube hole, and
wherein the positioning portion is a tapered portion in which an inner diameter of the tube portion gradually decreases in size from a first inner diameter larger than a diameter of the impact ball to a second inner diameter smaller than the diameter of the impact ball from one side in the axial direction toward the other side in the axial direction.

14. A restitution coefficient measuring device measuring a restitution coefficient of a measuring object, the restitution coefficient measuring device comprising:
a holder that holds a spherical impact ball colliding with the measuring object;
a shooting mechanism for shooting the impact ball held by the holder from the holder toward the measuring object;
a speed measuring unit that measures both a collision speed at which the impact ball collides with the measuring object and a restitution speed at which the impact ball bounces from the measuring object; and
a computing unit that calculates the restitution coefficient on the basis of the restitution speed with respect to the collision speed,
wherein the holder has a tube portion having a tubular shape for holding the impact ball in a tube hole,
wherein the shooting mechanism has a shooting member disposed on the other side in an axial direction from the impact ball and capable of moving in the tube hole in the axial direction, a energizing portion applying a energizing force to the shooting member toward one side in the axial direction, and a restriction portion restricting movement of the shooting member to one side in the axial direction,
wherein the shooting member has a small diameter portion on one side in the axial direction, a large diameter portion having a larger diameter than the small diameter portion on the other side in the axial direction from the small diameter portion, and a stepped portion which is a step generated due to a difference between the diameters of the small diameter portion and the large diameter portion, and
wherein the restriction portion is an engagement member engaging with the stepped portion.

15. The restitution coefficient measuring device according to claim 14,
wherein the tube portion has a penetration hole penetrating the tube portion from a side outward in a radial direction to a side inward in the radial direction on the other side in the axial direction from the impact ball and one side in the axial direction from the shooting member.

16. A hardness measuring device measuring a hardness of a measuring object, the hardness measuring device comprising:
a holder that bolds a spherical impact ball colliding with the measuring object with an elastic member;
a shooting mechanism for shooting the impact ball held by the holder from the holder toward the measuring object;
a speed measuring unit that measures both a collision speed at which the impact ball collides with the measuring object and a restitution speed at which the impact ball bounces from the measuring object; and
a computing unit that calculates the hardness on the basis of the restitution speed with respect to the collision speed,
wherein a hole for bleeding air is bored on any side surface of the holder, and
wherein the elastic member is an independent member replaceable with respect to the holder and is disposed at an end portion of the holder in an axial direction.

17. A hardness measuring device measuring a hardness of a measuring object, the hardness measuring device comprising:
a holder that holds a spherical impact ball colliding with the measuring object;
a shooting mechanism for shooting the impact ball held by the holder from the holder toward the measuring object;
a speed measuring unit that measures both a collision speed at which the impact ball collides with the measuring object and a restitution speed at which the impact ball bounces from the measuring object; and
a computing unit that calculates the hardness on the basis of the restitution speed with respect to the collision speed,
wherein the holder has a tube portion having a tubular shape for holding the impact ball in a tube hole,
wherein the shooting mechanism leas a shooting member disposed on the other side in an axial direction from the impact ball and capable of moving in the tube hole in the axial direction, a energizing portion applying a energizing force to the shooting member toward one side in the axial direction, and a restriction portion restricting movement of the shooting member to one side in the axial direction,
wherein the shooting member has a small diameter portion on one side in the axial direction, a large diameter portion having a larger diameter than the small diameter portion on the other side in the axial direction from the small diameter portion, and a stepped portion which is a step generated due to a difference between the diameters of the small diameter portion and the large diameter portion, and
wherein the restriction portion is an engagement member engaging with the stepped portion.
